(19) United States
(12) Patent Application Publication
(10) Pub. No.: US 2023/0128461 A1
(43) Pub. Date: Apr. 27, 2023

(54) INFORMATION PROCESSING APPARATUS AND COMPUTER-READABLE MEDIUM

(71) Applicant: Shunsuke KUDO, Kanagawa (JP)

(72) Inventor: Shunsuke KUDO, Kanagawa (JP)

(73) Assignee: Ricoh Company, Ltd., Tokyo (JP)

(21) Appl. No.: 17/963,002

(22) Filed: Oct. 10, 2022

(30) Foreign Application Priority Data
Oct. 25, 2021 (JP) 2021-173953

(51) Int. Cl. A61B 5/107; A61B 5/245; A61B 5/06

(52) U.S. Cl. CPC A61B 5/107; A61B 5/245; A61B 5/064

(57) ABSTRACT

An information processing apparatus includes an information acquisition unit and an estimation unit. The information acquisition unit is configured to acquire biological information on a measurement target portion of a measurement target person, the biological information being measured by a biological information measurement apparatus. The estimation unit is configured to estimate a position and a size of the measurement target portion using positional information on a plurality of locations in the measurement target portion and complementary positional information.

### CROSS-REFERENCE TO RELATED APPLICATIONS

The present application claims priority under 35 U.S.C. § 119 to Japanese Patent Application No. 2021-173953, filed on Oct. 25, 2021. The contents of which are incorporated herein by reference in their entirety.

### BACKGROUND OF THE INVENTION

### 1. Field of the Invention

The present invention relates to an information processing apparatus and a computer-readable medium.

### 2. Description of the Related Art

Conventionally, in a biological information measurement apparatus, such as a magnetoencephalography (MEG) measurement apparatus, a jamming signal or artifact as a noise that is measured simultaneously disturbs an analysis, and therefore need to be removed. Therefore, a noise removal technology using spatial separation for removing a jamming signal while maintaining a target signal that is generated from a measurement region by using information from a plurality of sensors and positional information on a head portion has been disclosed, for example.

Japanese Laid-open Patent Publication No. H09-253065 discloses a technology for estimating a spherical model (a position and a size) of a head portion by using a three-dimensional coordinate point, such as a marker coil (magnetic generation element), to achieve a sufficient noise removal effect when the positional information on the head portion is not acquired by Magnetic Resonance Imaging (MRI) or the like.

However, the conventional spherical model estimation method can estimate a spherical model with accuracy by using a large number of three-dimensional coordinate points, but has a problem that the accuracy is largely reduced if a spherical model is estimated using three to five marker coils that are practically adopted.

### SUMMARY OF THE INVENTION

According to an aspect of the present invention, an information processing apparatus includes an information acquisition unit and an estimation unit. The information acquisition unit is configured to acquire biological information on a measurement target portion of a measurement target person, the biological information being measured by a biological information measurement apparatus. The estimation unit is configured to estimate a position and a size of the measurement target portion using positional information on a plurality of locations in the measurement target portion and complementary positional information.

### BRIEF DESCRIPTION OF THE DRAWINGS

FIG. 1 is a diagram illustrating a configuration example of an information processing system according to a first embodiment;

FIG. 2 is a diagram illustrating a head portion as a measurement target of a measurement target person;

FIG. 3 is a block diagram illustrating an example of a hardware configuration of the information processing apparatus;

FIG. 4 is a block diagram illustrating a noise removal function of the information processing apparatus;

FIG. 5 is a flowchart schematically illustrating the flow of a spherical model estimation method;

FIG. 6 is a diagram illustrating an example of calculation of pseudo marker coordinates;

FIGS. 7A to 7E are diagrams illustrating a specific example of spherical model estimation;

FIGS. 8A to 8E are diagrams illustrating an effect achieved by the spherical model estimation;

FIG. 9 is a block diagram illustrating a noise removal function of an information processing apparatus according to a second embodiment; and

FIGS. 10A and 10B are diagrams illustrating a specific example of spherical model estimation.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to estimate the position and the size of the measurement target portion of the measurement target person with accuracy.

Embodiments of an information processing apparatus, a program, and a position estimation method will be described in detail below with reference to the accompanying drawings.

### First Embodiment

System Configuration

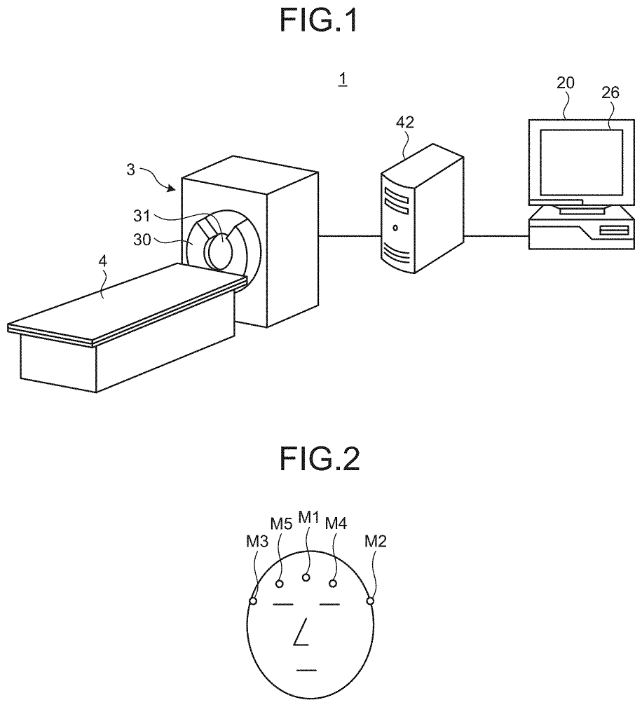

FIG. 1 is a diagram illustrating a configuration example of an information processing system 1 according to a first embodiment. In FIG. 1, the information processing system 1 measures and displays a plurality of kinds of biological signals, such as a magnetoencephalography (MEG) signal and an electroencephalography (EEG) signal. The information processing system 1 includes a measurement apparatus 3, a data recording server 42, and an information processing apparatus 20. The information processing apparatus 20 includes a monitor display 26 that displays signal information (biological information) that is obtained through measurement and an analysis result. In this example, the data recording server 42 and the information processing apparatus 20 are illustrated as separate apparatuses, but at least a part of the data recording server 42 may be incorporated in the information processing apparatus 20.

A measurement target person lies down on a measurement table 4 with face up while mounting electrodes (or sensors) for electroencephalography on his/her head, and inserts a measurement target portion (head portion) in a hollow 31 of a dewar 30 of the measurement apparatus 3. The dewar 30 is a holding container in an extremely low temperature environment using liquid helium, and a large number of magnetic sensors for magnetoencephalography are arranged inside the hollow 31 of the dewar 30. The magnetic sensors are high-sensitive superconducting quantum interference device (SQUID) sensors. In general, the dewar 30 in which the magnetic sensors are incorporated and the measurement table 4 are arranged in a magnetic shielding room, but illustration of the magnetic shielding room is omitted for the sake of convenience.

The measurement apparatus 3 collects electroencephalography signals from the electrodes and magnetoencephalography signals from the magnetic sensors, and outputs the collected biological signals (biological information) to the data recording server 42. The electroencephalography signal is a signal that represents electrical activity of a nerve cell (ion charge flow that occurs in dendrites of a neuron at the time of synaptic transmission) as a voltage value between the electrodes. The magnetoencephalography signal is a signal that represents minute magnetic field variation that occurs due to electrical activity of a brain.

The biological information that is recorded in the data recording server 42 is read, displayed, and analyzed by the information processing apparatus 20.

As described above, the measurement apparatus 3 is a biological information measurement apparatus, such as a magnetoencephalography (MEG) measurement apparatus (magnetoencephalogram), and functions as a multi-channel measurement apparatus. When positions and orientations of a large number of magnetic sensors for magnetoencephalography are already known and if a signal source located close to a group of the magnetic sensors is presumed, the measurement apparatus 3 as the multi-channel measurement apparatus is able to calculate response values of the magnetic sensors.

FIG. 2 is a diagram illustrating a head portion as a measurement target of a measurement target person. As illustrated in FIG. 2, marker coils M1, M2, M3, M4, and M5 as positioning Fiducial Points (FPs) are attached to the head portion as a measurement target portion of the measurement target person. More specifically, the marker coil M1 is attached to a nasal point or a center of a forehead above a nose, the marker coils M2 and M3 are attached to left and right ears, and the marker coils M4 and M5 are attached to left and right portions in the forehead across the nasal point. For example, the marker coils M1 to M5 are magnetic field generation elements that generate magnetic fields by application of voltage.

The measurement apparatus 3 measures positions of the marker coils on the basis of the magnetic fields that are generated by the marker coils at the time of measurement. The positions of the FPs that are represented by the MEG coordinate system and that are acquired as described above are used to calculate a coordinate transformation matrix for transformation into the coordinate system of a Magnetic Resonance Imaging (MRI) image when a measurement result obtained by the measurement apparatus 3 is superimposed on the MRI image of the measurement target person.

Meanwhile, in the present embodiment, the marker coils are used to calculate the coordinate transformation matrix between the coordinate system of the MRI image and the MEG coordinate system, but embodiments are not limited to this example, and an apparatus, such as a digitizer, that measures a three-dimensional coordinate position may be adopted.

The information processing apparatus 20 displays waveforms of the magnetoencephalography signals obtained from the plurality of magnetic sensors and waveforms of the electroencephalography signals obtained from the plurality of electrodes on the same time axis in a synchronized manner.

In addition, the information processing apparatus 20 performs a marker position estimation process and a spherical model estimation process when removing a jamming signal or artifact as a noise that is measured simultaneously by the measurement apparatus 3.

Hardware Configuration

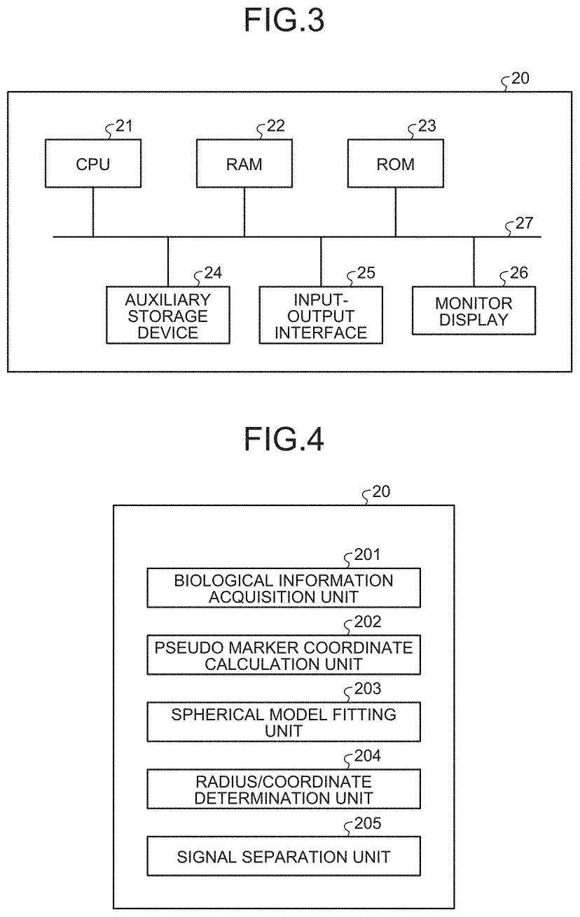

FIG. 3 is a block diagram illustrating an example of a hardware configuration of the information processing apparatus 20. The information processing apparatus 20 includes a central processing unit (CPU: a processor) 21, a random access memory (RAM) 22, a read only memory (ROM) 23, an auxiliary storage device 24, an input-output interface 25, and the monitor display (display device) 26. The CPU 21, the RAM 22, the ROM 23, the auxiliary storage device 24, the input-output interface 25, and the monitor display (display device) 26 are connected to one another via a bus 27.

The RAM 22 is used as a work area for the CPU 21, and may include a non-volatile RAM for storing a main control parameter and the biological information acquired from the data recording server 42. The ROM 23 stores therein a basic input-output program or the like. An information processing program of the present invention may be stored in the ROM 23.

The auxiliary storage device 24 is a storage device, such as a Solid State Drive (SSD) or a Hard Disk Drive (HDD), and stores therein, for example, an information processing program for controlling operation of the information processing apparatus 20, various kinds of data needed for the operation of the information processing apparatus 20, a file, and the like. The auxiliary storage device 24 stores therein a marker position estimation program and a spherical model estimation program.

The CPU 21 controls the entire operation of the information processing apparatus 20 and performs various kinds of information processing. The CPU 21 also executes the information processing program that is stored in the ROM 23 or the auxiliary storage device 24, and controls operation of displaying a measurement recording screen and an analysis screen.

The CPU 21 performs a marker position estimation process of obtaining three-dimensional position coordinates of the marker coils and pseudo marker coordinates of the position of the measurement target portion (head portion) of the measurement target person in the measurement apparatus 3, from acquired data of the marker coils in accordance with the marker position estimation program that is stored in the auxiliary storage device 24. The CPU 21 performs a spherical model estimation process of estimating a spherical model (position and size) of the measurement target portion (head portion) of the measurement target person, from the calculated three-dimensional position coordinates of the marker coil and the calculated pseudo marker coordinates of the measurement target portion (head portion) of the measurement target person in the measurement apparatus 3 in accordance with the spherical model estimation program that is stored in the auxiliary storage device 24.

The input-output interface 25 includes both of a user interface, such as a touch panel, a keyboard, a display screen, or an operation button, and a communication interface for inputting information from various sensors or the data recording server 42 and outputting analysis information to a different electronic apparatus.

The monitor display 26 displays the measurement recording screen and the analysis screen, and updates the screens in accordance with input-output operation that is performed via the input-output interface 25.

The information processing program (including the marker position estimation program and the spherical model estimation program) that is executed by the information processing apparatus 20 of the present embodiment is recorded in a computer readable recording medium, such as a compact disk-ROM (CD-ROM), a flexible disk (FD), a CD-recordable (CD-R), or a digital versatile disk (DVD), in a computer-installable or a computer-executable file format.

Furthermore, the information processing program (including the marker position estimation program and the spherical model estimation program) that is executed by the information processing apparatus 20 of the present embodiment may be stored in a computer that is connected to a network, such as the Internet, and may be provided by download via the network. Moreover, the information processing program (including the marker position estimation program and the spherical model estimation program) that is executed by the information processing apparatus 20 of the present embodiment may be provided or distributed via a network, such as the Internet.

Furthermore, the information processing program (including the marker position estimation program and the spherical model estimation program) that is executed by the information processing apparatus 20 of the present embodiment may be provided by being incorporated in a ROM or the like in advance.

Functional Configuration

A function to remove a jamming signal or artifact, which is a noise that is measured simultaneously by the measurement apparatus 3, among functions of the information processing apparatus 20 of the present embodiment will be described below. FIG. 4 is a block diagram illustrating a noise removal function of the information processing apparatus 20.

The information processing apparatus 20 includes a biological information acquisition unit 201, a pseudo marker coordinate calculation unit 202, a spherical model fitting unit 203, a radius/coordinate determination unit 204, and a signal separation unit 205.

The biological information acquisition unit 201, the pseudo marker coordinate calculation unit 202, the spherical model fitting unit 203, the radius/coordinate determination unit 204, and the signal separation unit 205 are implemented by causing the CPU 21 to read the information processing program (including the marker position estimation program and the spherical model estimation program) that is stored in the ROM 23 or the auxiliary storage device 24 and execute the information processing program.

The biological information acquisition unit 201 acquires, from the data recording server 42, a biological signal (biological information) that is sensor information on the measurement target portion (head portion) of the measurement target person and that is measured by the measurement apparatus 3. The biological information acquisition unit 201 is an information acquiring means. The biological information acquisition unit 201 stores the biological signal (biological information) that is the sensor information on the measurement target portion (head portion) of the measurement target person and that is measured by the measurement apparatus 3 in the RAM 22.

The pseudo marker coordinate calculation unit 202 calculates the pseudo marker coordinates of the position of the measurement target portion (head portion) of the measurement target person in the measurement apparatus 3. The pseudo marker coordinate calculation unit 202 is a pseudo marker coordinate calculating means.

The spherical model fitting unit 203 estimates, as a spherical model, a position and a size of the measurement target portion (head portion) of the measurement target person by using positional information on a plurality of locations in the measurement target portion (head portion) of the measurement target person and complementary positional information. The spherical model fitting unit 203 is an estimating means. More specifically, the spherical model fitting unit 203 adds, as the complementary positional information, the pseudo marker coordinates that are calculated by the pseudo marker coordinate calculation unit 202, and estimates the spherical model (position and size) of the measurement target portion (head portion) of the measurement target.

The radius/coordinate determination unit 204 determines a radius and center coordinates of the estimated spherical model.

The signal separation unit 205 performs spatial separation and removes a jamming signal and an artifact component on the basis of the spherical model for which the radius and the center coordinates are determined.

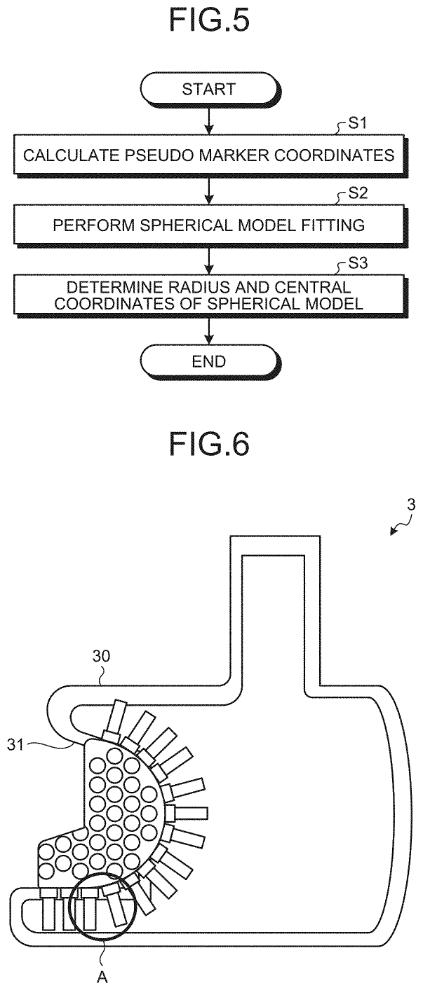

The flow of a spherical model estimation method for approximating the head portion (brain) of the measurement target person with a sphere will be described below. FIG. 5 is a flowchart schematically illustrating the flow of the spherical model estimation method.

As described above, it is often the case that the positions of the marker coils M1 to M5 that are used for positioning and that are attached to the head portion of the measurement target person are generally set at anatomical reference points, such as a forehead, a nasal point, and parotid portions. When the three-dimensional coordinate points of the marker coils as described above are used for estimation of the spherical model of the head portion, and if the number of the marker coils is three to five as adopted in the practical case, there is a problem in that the accuracy is largely reduced. In addition, when fitting is performed, there is a problem in that estimation accuracy is reduced or estimation is impossible only with the three to five marker coils as adopted in the practical case.

To cope with this, as illustrated in FIG. 5, in the present embodiment, the pseudo marker coordinate calculation unit 202 calculates, as pseudo marker coordinates, a position at which it is estimated that a back of the head comes into contact with the dewar 30 of the measurement apparatus 3, as a control point at the back of the head to which the marker coils M1 to M5 are not attachable, from coordinates and design values of the magnetic sensors of the measurement apparatus 3 (Step S1). Further, the pseudo marker coordinate calculation unit 202 adds the calculated position as a pseudo marker.

FIG. 6 is a diagram illustrating an example of calculation of the pseudo marker coordinates. For example, as illustrated in FIG. 6, the pseudo marker coordinate calculation unit 202 assumes that the back of the head comes into contact with a position of a magnetic sensor indicated by A in the measurement apparatus 3 in FIG. 6, and calculates pseudo marker coordinates of the position at which it is assumed that the head portion of the measurement target person actually comes into contact with the magnetic sensor, from the coordinates of the sensor and a thickness of the dewar 30 of the measurement apparatus 3.

Meanwhile, it may be possible to classify the magnetic sensors of the measurement apparatus 3 into a group for each area, and the pseudo marker coordinate calculation unit 202 may identify the sensors at the position of the back of the head from information on the classified groups. Furthermore, the pseudo marker coordinate calculation unit 202 may identify a sensor at a position other than the back of the head of the measurement target person without being limited to the back of the head as long as the head portion of the measurement target person comes into contact with any of the magnetic sensors.

Subsequently, the spherical model fitting unit 203 solves an optimization problem for performing fitting with a sphere by including the pseudo marker coordinates calculated at Step S1 in the coordinates of the marker coils (Step S2). Meanwhile, the spherical model fitting unit 203 uses a general optimization solution, such as the least squares method, for the spherical model fitting; however, it may be possible to use a machine learning method or the like. Furthermore, the sphere that is used by the spherical model fitting unit 203 for the fitting need not always be a single sphere, but may be a plurality of spheres (Overlapping-sphere Model) or a sphere of a standard brain. The sphere is not limited to the examples as described above.

Subsequently, the radius/coordinate determination unit 204 determines the radius and the center coordinates of the spherical model (Step S3). This process is performed because the radius of the spherical model calculated at Step S2 is larger than an actual size of the brain because the marker coils that are attached to the head portion of the measurement target person are used as references. Therefore, the radius/coordinate determination unit 204 reduces the radius of the spherical model calculated at Step S2 by a predetermined percentage, and sets the reduced radius as a radius of the spherical model that approximates the size of the brain. Meanwhile, the positions of the marker coils attached to the head portion of the measurement target person in this method need not always be set at the anatomical reference points.

A specific example of spherical model estimation will be described below.

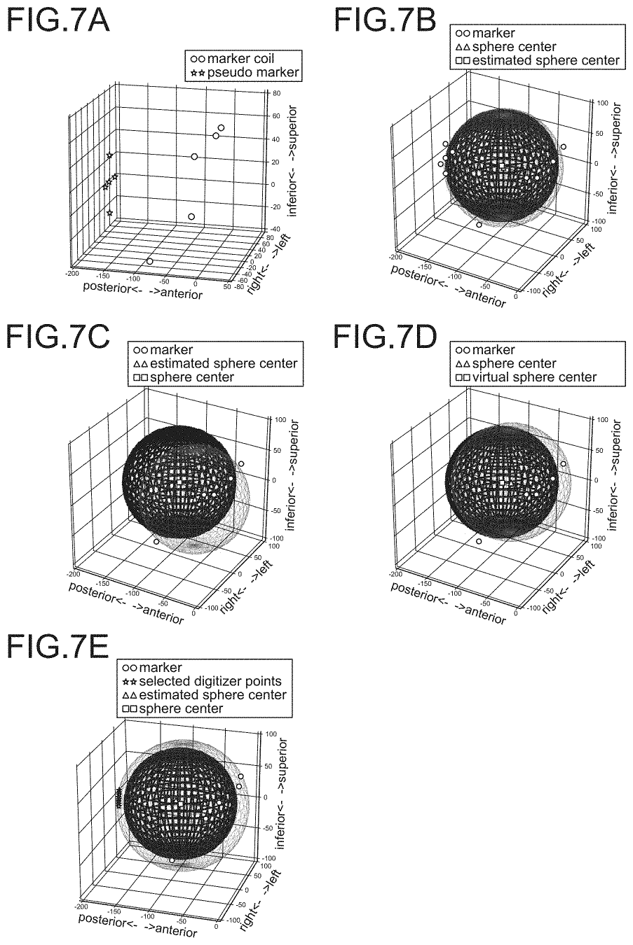

FIGS. 7A to 7E are diagrams illustrating a specific example of the spherical model estimation. Circles illustrated in FIG. 7A indicate results of the positions of the marker coils attached at five positions, such as left and right parotid portions and left, right, and center portions on the forehead, in the head portion of the measurement target person, which are acquired by using the measurement apparatus 3. When the marker coils are used in the measurement apparatus 3 that is the magnetoencephalography (MEG) measurement apparatus, it is often the case that a marker coil is not attached to the back of the head. In contrast, star marks illustrated in FIG. 7A indicate positions of pseudo marker coordinates that are obtained from the magnetic sensors of the measurement apparatus 3 and the design values of the magnetic sensor. With this configuration, it is possible to complement information on the back of the head of the measurement target person, and improve accuracy of the estimated spherical model.

FIG. 7B and FIG. 7C illustrate estimation results of spherical models in a case where the pseudo marker coordinates are present and in a case where the pseudo marker coordinates are absent. In both of FIG. 7B and FIG. 7C, spheres illustrated by black bold lines are spherical models that are generated from positional alignment with actual MRI and that represent accurate positions of the brain, whereas spheres illustrated by gray lines are spherical models that are estimated from the positions of the marker coils. Meanwhile, correction of the radius as described above with reference to Step S3 in FIG. 5 is not performed for the sake of illustration.

As illustrated in FIG. 7C, the spherical model that is estimated from only the positions of the marker coils may largely be deviated forward by being pulled by the marker coils attached to the forehead of the measurement target person. In contrast, the spherical model in which the pseudo marker coordinates as illustrated in FIG. 7B are added is less deviated than the spherical model illustrated in FIG. 7C. Therefore, it can be understood that adding the pseudo marker coordinates is effective.

FIG. 7D illustrates a spherical model that is obtained by a method of setting a virtual spherical model that is calculated from the positions of centers of gravity of magnetoencephalography sensors as used in the conventional technology. A size and a position of a sphere in the spherical model illustrated in FIG. 7D are deviated from the spherical model in which the pseudo marker coordinates are added as illustrated in FIG. 7B. Therefore, it can be understood that estimation of the spherical model by adding the pseudo marker coordinates is effective.

Meanwhile, in the present embodiment, the pseudo marker coordinates are obtained from the magnetic sensors of the measurement apparatus 3 that is a magnetoencephalography (MEG) measurement apparatus and the design values of the magnetic sensors, but it may be possible to use three-dimensional coordinate points that are measured by a digitizer or the like as illustrated in FIG. 7E as the pseudo marker coordinates. In this case, the three-dimensional coordinate points acquired from the digitizer need not cover the entire head portion of the measurement target person, and it is sufficient to add several points in at least a portion that is not covered by the marker coils (in the posterior direction in FIG. 7E). Furthermore, it may be possible to obtain surface coordinates from MRI of a standard brain and use the surface coordinates as a substitute for the pseudo marker coils.

Effects of the spherical model estimation will be described below.

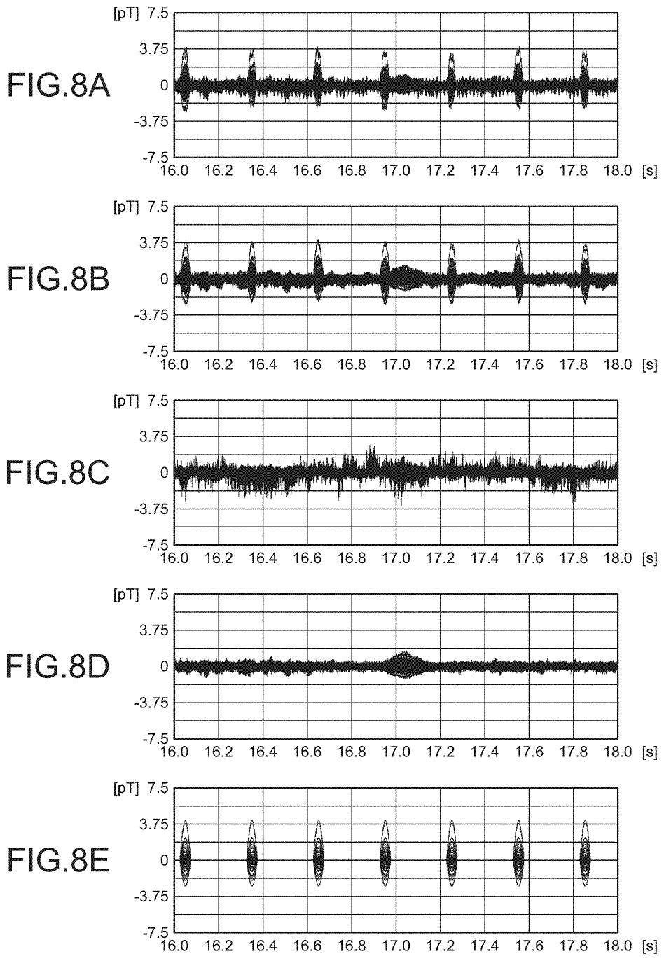

FIGS. 8A to 8E are diagrams illustrating the effects of the spherical model estimation. FIGS. 8A to 8E illustrate results that are obtained by performing, with respect to noise data that is recorded by the measurement apparatus 3 as a 160-channel magnetoencephalography (MEG) measurement apparatus, Double Signal Subspace Projection (DSSP) for obtaining a partial space of a time domain of a noise, such as a jamming signal, by embedding an artificial brain signal as illustrated in FIG. 8E into an occipital lobe. DSSP is a method that is effective to remove noise of magnetoencephalograph, but, to bring out the performance, it is necessary to accurately set the brain region; therefore, effects of setting a spherical model according to the present invention will be described below using DSSP as an example.

FIG. 8A illustrates a result that is obtained by performing DSSP on a spherical model that is generated using an MRI image. The result indicates an ideal condition because an accurate position of the head portion is obtained by MRI. FIG. 8B illustrates a result that is obtained by performing DSSP on the spherical model that is generated using the coordinates of the marker coils and the coordinates the pseudo marker coils without using MRI as illustrated in FIG. 7B. According to FIG. 8A and FIG. 8B, it can be understood that noise is substantially removed and the artificial brain signal is maintained as compared to the artificial signal illustrated in FIG. 8E.

In contrast, FIG. 8C illustrates a result that is obtained by performing DSSP on the spherical model illustrated in FIG. 7C, and FIG. 8D illustrates a result that is obtained by performing DSSP on the spherical model illustrated in FIG. 7D. According to FIG. 8C and FIG. 8D, the embedded artificial brain signal is eliminated as compared to the artificial signal illustrated in FIG. 8E.

In other words, if the spherical model is deviated as illustrated in FIG. 7C and FIG. 7D, even a signal that is normally generated from the brain is removed. Therefore, it is important to estimate the spherical model with improved accuracy, and the method of the present embodiment is effective especially in the case where MRI is not present.

As described above, according to the present embodiment, the pieces of positional information on the marker coils that are used for positioning and that are attached to the measurement target portion (head portion) of the measurement target person are obtained by using the measurement apparatus 3, and the position and the size of the measurement target portion (head portion) of the measurement target person is obtained through spherical model fitting using the least squares method, by using the pieces of positional information on the marker coils and the complementary positional information (pseudo marker coordinates). With this configuration, even if an MRI image or the like is not provided, it is possible to achieve noise removal performance similar to the case in which the image is provided, so that it is possible to accurately estimate the position and the size of the measurement target portion (head portion) of the measurement target person by attaching positioning marker coils provided with a general magnetoencephalography measurement apparatus to several portions in the measurement target portion (head portion) of the measurement target person and obtaining pieces of positional information on the marker coils, without adding any special device.

### Second Embodiment

A second embodiment will be described below.

The second embodiment is different from the first embodiment in that an appropriate constraint condition is added to the least squares method at the time of the spherical model fitting. In the following description of the second embodiment, explanation of the same components as those of the first embodiment will be omitted, and differences from the first embodiment will be described.

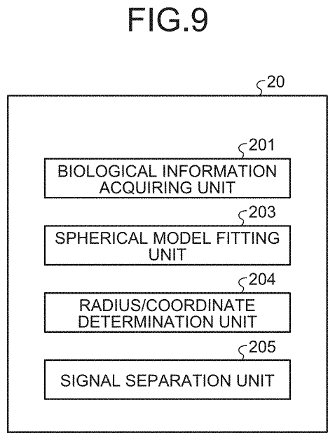

FIG. 9 is a block diagram illustrating a noise removal function of the information processing apparatus 20 according to the second embodiment. As illustrated in FIG. 9, the information processing apparatus 20 according to the second embodiment does not include the pseudo marker coordinate calculation unit 202 illustrated in FIG. 4.

Further, the spherical model fitting unit 203 sets a constraint condition on positional information on a plurality of locations in the measurement target portion (head portion) of the measurement target person, and estimates the position and the size of the measurement target portion (head portion) of the measurement target person.

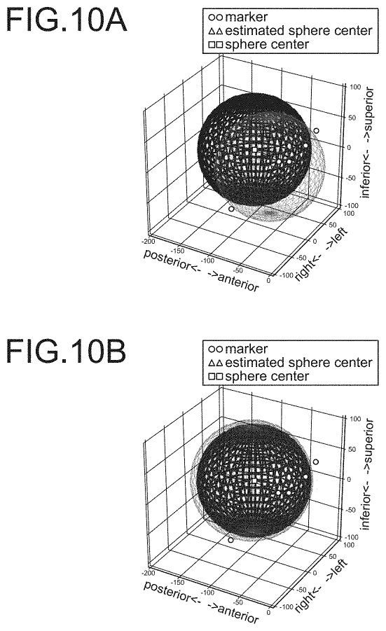

FIGS. 10A and 10B are diagrams illustrating a specific example of spherical model estimation. FIG. 10A illustrates, similar to the case illustrated in FIG. 7C, an estimation result of a spherical model in the case where the pseudo marker coordinates are not provided. As illustrated in FIG. 10A, if the spherical model is estimated by only the three-dimensional position coordinates of the marker coils, the estimation result may be largely degraded depending on the positions of the marker coils. To cope with this, in the present embodiment, the spherical model fitting unit 203 sets an appropriate constraint condition on the least squares method at the time of the spherical model fitting in order to estimate a sphere as illustrated by a gray line in FIG. 10B.

For example, a constraint condition in a case where a total of five points, such as left and right parotid portions and left, right, and center portions on the forehead, in the head portion of the measurement target person are obtained as the three-dimensional coordinate points will be described below.

A first constraint condition is to correct left and right deviation of a sphere that is estimated from the positions of the anatomical reference points. The first constraint condition imposes a constraint on a sphere center such that the sphere center is located in a plane that is perpendicular to a segment connecting the left and right parotid portions and that includes the position of the center of the forehead.

A second constraint condition constrains anterior and posterior deviation of the estimated sphere. The second constraint condition imposes a constraint on an angle between a vector of a perpendicular line that is extended from the center of the forehead to the segment connecting the left and right parotid portions and a vector that connects the foot of the perpendicular line and the center of the estimated sphere such that the angle falls within a certain range.

In this manner, the spherical model fitting unit 203 sets an appropriate constraint condition on the least squares method at the time of fitting, so that it is possible to perform estimation even with only the five marker coils, and it is possible to perform estimation using the characteristics of the anatomical reference points.

Furthermore, the constraint condition may be set so as to determine a range in which the center of the estimated sphere may be present from the positions of the magnetic sensors of the measurement apparatus 3 that is the magnetoencephalography (MEG) measurement apparatus, the positions of the marker coils, and the three-dimensional coordinate points of the digitizer, and add a linear constraint. For example, there are a method of assuming that the center position of the sphere is located in the vicinity of a position of the center of gravity of a magnetic sensor of the measurement apparatus 3 that is a magnetoencephalography (MEG) measurement apparatus, and making limitation to a range so as to adopt the range of several tens of millimeters in each of the x coordinate, the y coordinate, and the z coordinate from the position of the center of gravity of the magnetic sensor of the measurement apparatus 3 that is a magnetoencephalography (MEG) measurement apparatus, or a method of imposing a limitation on positions to prevent the estimated sphere from digging into the dewar 30.

As described above, according to the present embodiment, the pieces of positional information on the marker coils that are used for positioning and that are attached to the measurement target portion (head portion) of the measurement target person are obtained, a constraint condition on the pieces of positional information is set, and the position and the size of the measurement target portion (head portion) of the measurement target person are obtained through spherical model fitting using the least squares method. With this configuration, even if an MRI image or the like is not provided, it is possible to achieve noise removal performance similar to the case in which the image is provided, so that it is possible to accurately estimate the position and the size of the measurement target portion (head portion) of the measurement target person by attaching positioning marker coils provided with a general magnetoencephalography measurement apparatus to several portions in the measurement target portion (head portion) of the measurement target person and obtaining the pieces of positional information on the marker coils, without adding any special device.

Meanwhile, it may be possible to combine the setting of the constraint condition of the second embodiment with the addition of the pseudo marker coordinates described in the first embodiment.

Meanwhile, in each of the embodiments, the example is described in detail by taking a case in which the head portion is considered as the measurement target portion of the measurement target person, but embodiments are not limited to this example.

According to an embodiment, it is possible to estimate the position and the size of the measurement target portion of the measurement target person with accuracy.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. An information processing apparatus comprising:
an information acquisition unit configured to acquire biological information on a measurement target portion of a measurement target person, the biological information being measured by a biological information measurement apparatus; and
an estimation unit configured to estimate a position and a size of the measurement target portion using positional information on a plurality of locations in the measurement target portion and complementary positional information.

2. The information processing apparatus according to claim 1, wherein
the biological information measurement apparatus is configured to set a head portion of the measurement target person as the measurement target portion, and
the estimation unit is configured to estimate a spherical model of the head portion of the measurement target person.

3. The information processing apparatus according to claim 1, further comprising:
a pseudo marker coordinate calculation unit configured to calculate pseudo marker coordinates of a position in the measurement target portion in the biological information measurement apparatus, wherein
the estimation unit is configured to add the pseudo marker coordinates calculated by the pseudo marker coordinate calculation unit as the complementary positional information, to estimate the position and the size of the measurement target portion.

4. The information processing apparatus according to claim 3, wherein the pseudo marker coordinate calculation unit is configured to, in a case where the positional information on the plurality of locations in the measurement target portion is insufficient, virtually set a contact position of the measurement target portion based on coordinates of a sensor included in the biological information measurement apparatus, to be the pseudo marker coordinates.

5. The information processing apparatus according to claim 1, wherein the estimation unit is configured to set a constraint condition on the positional information on the plurality of locations in the measurement target portion, and estimate the position and the size of the measurement target portion.

6. An information processing apparatus comprising:
an information acquisition unit configured to acquire biological information on a measurement target portion of a measurement target person, the biological information being measured by a biological information measurement apparatus; and
an estimation unit configured to set a constraint condition on positional information on a plurality of locations in the measurement target portion, and estimate a position and size of the measurement target portion.

7. The information processing apparatus according to claim 6 wherein
the biological information measurement apparatus is configured to set a head portion of the measurement target person as the measurement target portion, and
the estimation unit is configured to estimate a spherical model of the head portion of the measurement target person.

8. A non-transitory computer-readable medium including programmed instructions that cause a computer to function as:
an information acquisition unit configured to acquire biological information on a measurement target portion of a measurement target person, the biological information being measured by a biological information measurement apparatus; and
an estimation unit configured to estimate a position and a size of the measurement target portion using positional information on a plurality of locations in the measurement target portion and complementary positional information.
